(19) United States
(12) Patent Application Publication
(10) Pub. No.: US 2007/0003577 A1
(43) Pub. Date: Jan. 4, 2007

(54) Purified trimeric S protein as vaccine against severe acute respiratory syndrome virus infections

(76) Inventors: Yiu Wing Kam, Hong Kong (HK); Francois Kien, Hong Kong (HK); Kid Chu, Hong Kong (HK); Anjeanette Roberts, Washington, DC (US); Kanta Subbarao, Washington, DC (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/475,237

(22) Filed: Jun. 27, 2006

Related U.S. Application Data
(60) Provisional application No. 60/694,460, filed on Jun. 28, 2005.

(51) Int. Cl. A61K 48/00; A61K 39/215

(52) U.S. Cl. 424/221.1; 514/44

(57) ABSTRACT

Immunogenic compositions and vaccines associated with the Spike protein of SARS Corona Virus (SARS CoV) are disclosed.

### RELATED APPLICATION

This application claims priority to Provisional Application No. 60/694,460, filed Jun. 28, 2005, the entire disclosure of which is hereby incorporated herein by reference.

### FIELD OF THE INVENTION

The invention is directed to the use of nucleic acids and polypeptides in immunogenic compositions, vaccines, or antiviral therapy.

### BACKGROUND OF THE INVENTION

A new infectious disease, known as severe acute respiratory syndrome (SARS), appeared in Guangdong province of southern China in 2002. SARS spread to 29 countries, affected a reported 8,098 people, and left 774 patients dead. (Stadler et al.) Although the SARS epidemic was contained by aggressive quarantine measures, there is no information on when, or if, SARS will re-emerge in the human population.

SARS is mainly characterized by flu-like symptoms, including high fevers exceeding 100.4° F., myalagia, dry nonproductive dyspnea, lymphopaenia, and infiltrate on chest radiography. (Stadler et al.) In 38% of all cases, the resulting pneumonia led to acute breathing problems requiring artificial respirators. The overall mortality rate was about 10%, but varied profoundly with age, as SARS appeared to be milder in the pediatric age group while the mortality rate in the elderly was as high as 50%.

SARS is caused by a previously unknown coronavirus (CoV), a diverse group of large, enveloped viruses that cause respiratory and enteric disease and in humans and animals. SARS CoV was isolated from FRhK-4 and Vero E6 cells that were inoculated with clinical specimens from patients, and macaques inoculated with this virus developed symptoms similar to those observed in human cases of SARS. To date, over 30 different SARS CoV have been isolated and sequenced.

SARS CoV contains an RNA genome of about 30 kB (Accession No. AY310120), and shares many characteristic features of coronaviruses. Nucleotides 1-72 contain a predicted RNA leader sequence preceding an untranslated region (UTR) spanning 192 nucleotides. Two overlapping open reading frames, which encompass approximately two-thirds of the genome (nucleotides 265-21485) are down stream of the UTR, and encode proteinases as well as the proteins required for replication and transcription (for a review see Stadler et al., 2004). The remaining 3′ part of the genome encodes four structural proteins that are arranged in the same order in all CoV: Spike, Envelope, Membrane glycoprotein, and Nucleocapsid protein. The structural protein region of the SARS CoV genome also encodes additional non-structural proteins known as ‘accessory genes’. Although the overall organization of the SARS CoV genome is similar to other coronaviruses, the amino acid conservation of the encoded proteins is usually low.

The Spike protein forms large surface projections that are characteristic of coronaviruses. Spike is heavily glycosylated and has 1,255 amino acids, containing an amino-terminal bulbous head adjacent to a stem, a single transmembrane region, and a short cytoplasmic tail.

Although β-interferon has been reported to interfere with the replication of the SARS virus in vitro, no licensed drug or vaccine is available. Moreover, large-scale screening of existing antivirals or big chemical libraries for potential replication inhibitors has not been very successful. It is also virtually impossible to confirm a SARS diagnosis in the primary care setting, as the sensitivity and specificity of available tests varies with time from onset of contact or symptoms (See Rainer et al.). At present, there are no easy, rapid, accurate tests for diagnosing SARS during the first week of illness, and none that will give a result within hours of sampling.

For these reasons, there is considerable need for effective means to treat or control SARS CoV infection.

### SUMMARY OF THE INVENTION

Accordingly, this invention aids in fulfilling these needs in the art.

This invention provides an immunogenic composition comprising at least one recombinant Spike protein having all of the epitopes of native Spike protein and free of other native SARS CoV components in an amount sufficient to induce an immunogenic or protective response in vivo, in association with a pharmaceutically acceptable carrier therefor. A vaccine composition of the invention comprises a neutralizing amount of the Spike polypeptide and a pharmaceutically acceptable carrier therefor.

The nucleic acid molecules that are used in the invention, which include DNA and RNA, are referred to herein as “Spike nucleic acids” or “Spike DNA”, and the amino acids encoded by these molecules are referred to herein as “Spike polypeptides” or “Spike protein.”

The present invention also pertains to vaccine compositions for immunizing humans and mammals against SARS CoV, comprising an immunogenic composition as described above in combination with one or more pharmaceutically compatible excipients (such as, for example, saline buffer), and optionally in combination with at least one adjuvant, such as aluminum hydroxide or a compound belonging to the muramyl peptide family.

Additional features and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1. Analysis of antibody-dependent enhancement of Spike-mediated viral entry. (A) Analysis of various cell lines for ADE of Spike-mediated viral entry. Lentiviral vectors with luciferase as reporter gene and pseudotyped with optimized SARS-CoV Spike were incubated with a 1:1000 dilution of sera from mice immunized with TriSpike (Post-dose 2). All infections were performed in triplicates. Data are presented as averages±standard deviations. (B) ADE in Raji B cells and neutralization in VeroE6 cells of Spike-mediated viral entry with various dilutions of sera from mice immunized with TriSpike (Post-dose 2). All infections were performed in triplicates. Data are presented as averages±standard deviations. (C) Same as (B) except that sera from hamsters immunized with TriSpike (Post-dose 3) were used.

FIG. 2. Replication of SARS-CoV in naive and immunized hamster lungs from intranasal challenge. (A) Hamsters were subcutaneously immunized with 2, 10 or 50 ug of TriSpike (on day 0, 21 and 42) at NIAID, NIH. Neutralizing antibody was measured from immunized hamsters. (B) Hamsters were inoculated intranasally with 10³TCID₅₀ of SARS-CoV on day 56 and lung homogenates were collected two days post challenge. Significant level (˜100000 fold) of viral replication reduction was obtained from hamster group (2 μg TriSpike) compared with control hamster. Further reduction to basal level of viral titer was obtained from hamster group number. (10 μg and 50 μg TriSpike). Virus titers in each groups of lung homogenates are the mean values calculated from four hamsters two days post challenge. Error bars indicated standard errors. *** indicated the value of p<0.001 in two-tailed t tests. Values were expressed as log₁₀TCID₅₀ per g of lung tissue.

### DETAILED DESCRIPTION OF THE INVENTION

Recombinant full-length trimeric S-protein covering all available epitopes of S protein is protective when administered with Alum adjuvant, which is authorised for human use. Although there is evidence of virus entry enhancing antibodies in vitro, no enhancement of virus replication has been observed in vivo in vaccinated animals.

The TriSpike vaccine of the invention is based on a purified protein which is less likely to induce undesirable side effects in the vaccinated subject compared to vaccines based on the entire SARS CoV (whole inactivated vaccine) genetic vectors (MVA, DNA). The TriSpike vaccine of the invention is less likely to induce unwanted immune responses against other viral proteins or co-purified cellular proteins. There is no risk of DNA integration or vector-induced toxicity.

This invention is useful for the protection of the general population in general and health care workers and animal handlers in wet markets in particular. Vaccination of civet cats in farms in southern China would reduce the chance of wild type SARS CoV amplification in this host and reduce the risk of wild type SARS CoV transmission to humans in close contact with these animals. Thus, the invention is useful in veterinary vaccine and human vaccine.

Nucleic acid sequences within the scope of the invention include isolated DNA and RNA sequences that encode Spike polypeptides. The polypeptides encoded by these nucleic acids are referred to herein as “Spike polypeptides” or “Spike proteins.” As used herein, these terms refer to a genus of polypeptides that further encompasses proteins having the amino acid sequence of Spike proteins, as well as those proteins and polypeptides having a high degree of similarity (at least 90% homology) with such amino acid sequences and which proteins and polypeptides are immunoreactive. In addition, “Spike polypeptides” and “Spike proteins” refer to those proteins encoded by nucleic acid molecules, which hybridize under conditions of high stringency to the nucleic acid strand complementary to the coding sequence of Spike proteins,.

The term “purified”, as used herein, means that the Spike polypeptides are essentially free of association with other proteins or polypeptides, for example, as a purification product of recombinant host cell culture or as a purified product from a non-recombinant source. The term “substantially purified”, as used herein, refers to a mixture that contains Spike polypeptides and is essentially free of association with other proteins or polypeptides, but for the presence of known proteins that can be removed using a specific antibody, and which substantially purified Spike polypeptides can be used as antigens. In a preferred embodiment of the invention, the Spike protein is free of native viron components.

A Spike polypeptide “variant” as referred to herein means a polypeptide substantially homologous to native Spike polypeptides, but which has an amino acid sequence different from that of native Spike polypeptides because of one or more deletions, insertions, or substitutions. The variant amino acid sequence preferably is at least 80% identical to a native Spike polypeptide amino acid sequence, most preferably at least 90% identical. The percent identity can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482,1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known.

Naturally occurring Spike polypeptide variants are also encompassed by the invention. Examples of such variants are proteins that result from alternate mRNA splicing events or from proteolytic cleavage of the Spike polypeptides. Variations attributable to proteolysis include, for example, differences in the termini upon expression in different types of host cells, due to proteolytic removal of one or more terminal amino acids from the Spike polypeptides. Variations attributable to frameshifting include, for example, differences in the termini upon expression in different types of host cells due to different amino acids.

As stated above, the invention provides isolated and purified, or homogeneous, Spike polypeptides, both recombinant and non-recombinant. Variants and derivatives of native Spike polypeptides that can be used as antigens can be obtained by mutations of nucleotide sequences coding for native Spike polypeptides. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

Recombinant expression vectors containing a nucleic acid sequence encoding Spike polypeptides can be prepared using well known methods. The expression vectors include a Spike DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the Spike DNA sequence. Thus, a promoter nucleotide sequence is operably linked to a Spike DNA sequence if the promoter nucleotide sequence controls the transcription of the Spike DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with Spike polypeptides can be incorporated into expression vectors.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982).

Suitable host cells for expression of Spike polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems can also be employed to produce Spike polypeptides using RNAs derived from DNA constructs disclosed herein.

It will be understood that the present invention is intended to encompass the use of the previously described proteins in isolated or purified form, whether obtained using the techniques described herein or other methods. In a preferred embodiment of this invention, the Spike polypeptides are substantially free of human tissue and human tissue components, nucleic acids, extraneous proteins and lipids, and adventitious microorganisms, such as bacteria and viruses. It will also be understood that the invention encompasses equivalent proteins having substantially the same biological and immunogenic properties. Thus, this invention is intended to cover serotypic variants of the proteins of the invention.

The invention provides immunogenic Spike polypeptides, and more particularly, protective polypeptides for use in the preparation of vaccine compositions against SARS CoV. These polypeptides can thus be employed as viral vaccines by administering the polypeptides to a mammal susceptible to SARS CoV infection. Conventional modes of administration can be employed. For example, administration can be carried out by oral, respiratory, or parenteral routes. Intradermal, subcutaneous, and intramuscular routes of administration are preferred when the vaccine is administered parenterally.

The major purpose of the immune response in a SARS CoV infected mammal is to inactivate the free SARS CoV and to eliminate SARS CoV infected cells that have the potential to release infectious virus. The B-cell arm of the immune response has the major responsibility for inactivating free SARS CoV virus. The principal manner in which this is achieved is by neutralization of infectivity. Another major mechanism for destruction of the SARS CoV infected cells is provided by cytotoxic T lymphocytes (CTL) that recognize viral Spike antigens expressed in combination with class I histocompatibility antigens at the cell surface. The CTLs recognize Spike polypeptides processed within cells from a Spike protein that is produced, for example, by the infected cell or that is internalized by a phagocytic cell. Thus, this invention can be employed to stimulate a B-cell response to Spike polypeptides, as well as immunity mediated by a CTL response following viral infection. The CTL response can play an important role in mediating recovery from primary SARS CoV infection and in accelerating recovery during subsequent infections.

The vaccine composition according to the present invention is advantageously prepared as an injectable form (either as liquid solution or suspension). However, solid forms suitable for solution in or suspension in, liquid prior injection may also be prepared.

In addition, if desired, the vaccine composition can contain minor amounts of auxiliary substances such as wetting or emulsifying agents, pH buffering agents, or adjuvants, which enhance the effectiveness of the vaccine.

The vaccine compositions of the invention are administered in a manner compatible with the dosage formulation, and in such amount as will be therapeutically effective and immunogenic. The quantity to be administered depends on the subject to be treated including, e.g., the capacity of the individual's immune system to induce an immune response.

The dosage of the vaccine will depend on the route of administration and will vary according to the age of the patient to be vaccinated and, to a lesser degree, the size of the person to be vaccinated.

The ability of the Spike polypeptides and vaccines of the invention to induce protective levels of neutralizing antibody in a host can be enhanced by emulsification with an adjuvant, incorporating in a liposome, coupling to a suitable carrier, or by combinations of these techniques. For example, the Spike polypeptides of the invention can be administered with a conventional adjuvant, such as aluminum phosphate and aluminum hydroxide gel, in an amount sufficient to potentiate humoral or cell-mediated immune response in the host. Similarly, the Spike polypeptides can be bound to lipid membranes or incorporated in lipid membranes to form liposomes. The use of nonpyrogenic lipids free of nucleic acids and other extraneous matter can be employed for this purpose.

Various methods for achieving adjuvant effect for the vaccine include the use of aluminum hydroxide or phosphate (alum), commonly used as 0.05 to 0.1 percent solution in phosphate buffered saline, admixture with synthetic polymers of sugars (Carbopol) used as 0.25% solution. Another suitable adjuvant compound comprises DDA (dimethyldioctadecyl-ammonium bromide), as well as immune modulating substances, such as lymphokines (e.g., IFN-gamma, IL-1, IL-2, and IL-12) or IFN-gamma inducer compounds, such as poly I:C.

The immunization schedule will depend upon several factors, such as the susceptibility of the host to infection and the age of the host. A single dose of the vaccine of the invention can be administered to the host or a primary course of immunization can be followed in which several doses at intervals of time are administered. Subsequent doses used as boosters can be administered as needed following the primary course.

The Spike proteins, polypeptides, and vaccines of the invention can be administered to the host in an amount sufficient to prevent or inhibit SARS CoV infection or replication in vivo. In any event, the amount administered should be at least sufficient to protect the host against substantial immunosuppression, even though SARS CoV infection may not be entirely prevented. An immunogenic response can be obtained by administering the Spike proteins or glycoproteins of the invention to the host in an amount of about 10 to about 500 micrograms antigen per kilogram of body weight, preferably about 50 to about 100 micrograms antigen per kilogram of body weight. The proteins and vaccines of the invention can be administered together with a physiologically acceptable carrier. For example, a diluent, such as water or a saline solution, can be employed.

The methods of treating include administering immunogenic compositions comprising Spike polypeptides, but compositions comprising nucleic acids encoding Spike polypeptides as well. Those of skill in the art are cognizant of the concept, application, and effectiveness of nucleic acid vaccines and nucleic acid vaccine technology as well as protein and polypeptide based technologies. The nucleic acid based technology allows the administration of nucleic acids encoding Spike polypeptides, naked or encapsulated, directly to tissues and cells without the need for production of encoded proteins prior to administration. The technology is based on the ability of these nucleic acids to be taken up by cells of the recipient organism and expressed to produce an immunogenic determinant to which the recipient's immune system responds. Typically, the expressed antigens are displayed on the surface of cells that have taken up and expressed the nucleic acids, but expression and export of the encoded antigens into the circulatory system of the recipient individual is also within the scope of the present invention. Such nucleic acid vaccine technology includes, but is not limited to, delivery of naked DNA and RNA and delivery of expression vectors encoding Spike polypeptides. Although the technology is termed “vaccine”, it is equally applicable to immunogenic compositions that do not result in a protective response. Such non-protection inducing compositions and methods are encompassed within the present invention.

Although it is within the present invention to deliver nucleic acids encoding Spike polypeptides and carrier molecules as naked nucleic acid, the present invention also encompasses delivery of nucleic acids as part of larger or more complex compositions. Included among these delivery systems are viruses, virus-like particles, or bacteria containing the nucleic acid encoding Spike polypeptides. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical's patents), which are incorporated by reference herein, and can be made and used without undue or excessive experimentation.

Protein based SARS vaccine can induce a neutralizing and protective antibody-dependent immune response after a single or double injection of Spike protein. Protein based vaccines present considerable safety advantages over vector-expressed (i.e., plasmid, MVA, Adeno) or whole inactivated virus vaccine.

The method of the invention includes administering any combination of the nucleic acids encoding Spike polypeptides, the proteins and polypeptides per se, with or without carrier molecules, to an individual. In embodiments, the individual is an animal, and is preferably a mammal, especially a primate. More preferably, the mammal is selected from the group consisting of a human, a mouse, a rat, a rabbit, a sheep, a dog, a cat, a bovine, a pig, and a horse. In an especially preferred embodiment, the mammal is a human.

This invention will be described in greater detail in the following Examples.

### EXAMPLE 1

### Preparation of TriSpike Vaccine for Mouse and Hamster Immunisation

The baby hamster kidney (BHK)-21 cell line was cultured at 37° C., 5% CO₂, in GMEM medium supplemented with 5% FCS, Hepes 20 mM, Tryptose-phosphate broth 10%, penicillin 100 U/ml and streptomycin 100 ug/ml. 14 hours post-infection/transfection with S-protein encoding Semliki Forest Virus vectors, BHK-21 cells were lysed (20 mM Tris-HCL 7.5, 150 mM NaCl, 2 mM EDTA, 1% Triton X-100) and incubated for 5 min on ice. The collected lysate was vortexed, incubated for another 15 min on ice prior to centrifugation at 13000 rpm for 15 min. Recombinant S-protein was immunoprecipitated from the supernatant using anti-FLAG M2 mAb-coated agarose beads (Sigma) overnight at 4° C. Subsequently beads were washed three times with 1× washing buffer (Sigma) and recombinant S-protein was eluted with 3× FLAG peptide according to the supplier's instructions (Sigma). Eluted recombinant S-protein was concentrated and impurities below a molecular weight of 100 kDa removed with centrifugal filter devices (Amicon) according to the supplier's instructions.

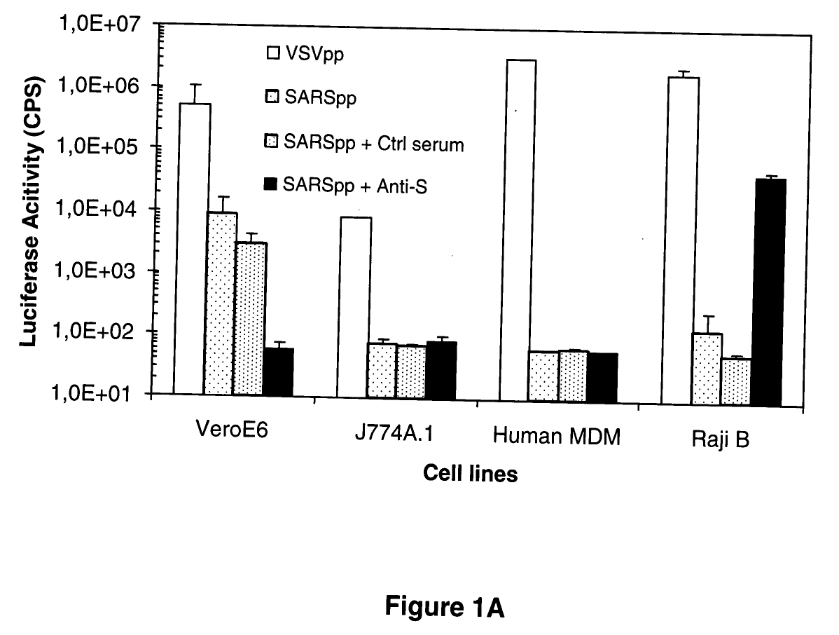

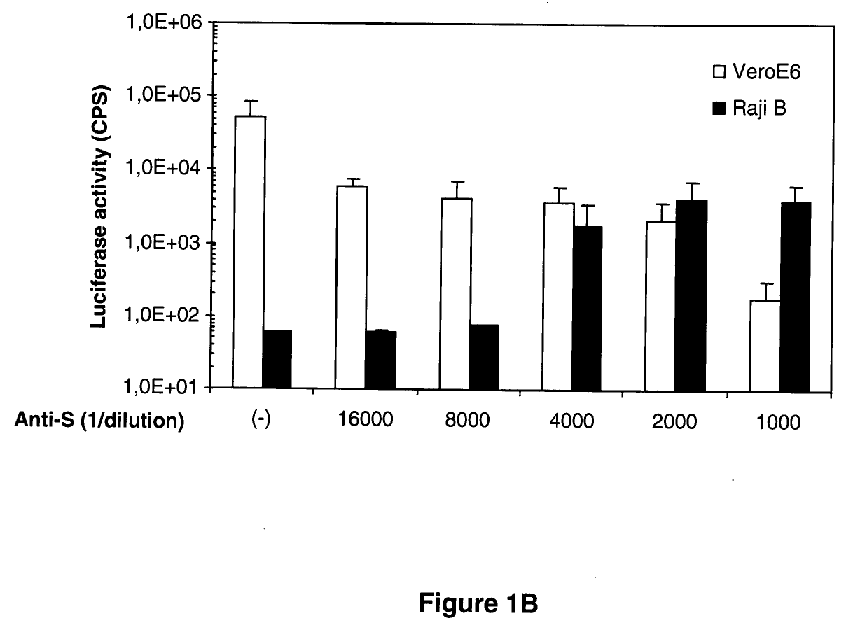

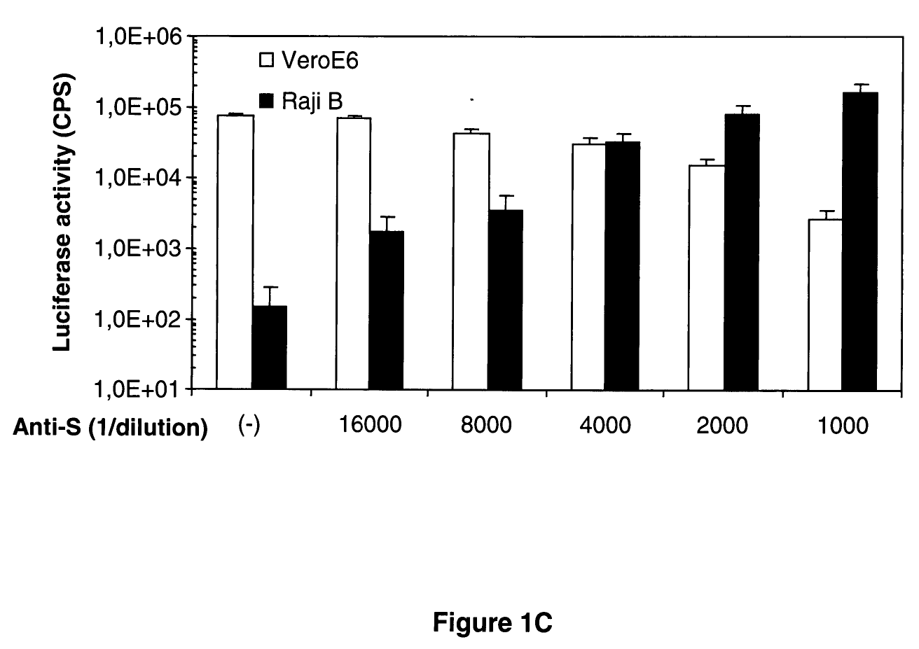

The quantity and quality of recombinant S-protein was assessed by SDS-PAGE and Western Blot using BAP-FLAG protein and microBSA methods as standards for protein quantification as previously described (Lozach et al., 2003, Staropoli et al., 2000). Briefly, protein samples were analyzed on 4-12% Bis-Tris SDS-PAGE gel (Invitrogen) under non-reducing conditions, except in experiments represented in FIG. 1 where different denaturing conditions were used as indicated. Proteins were transferred to PVDF membrane (Amersham Biosciences) and reacted with diluted mouse sera (1/500). After washing, the membrane was reacted with HRP-conjugated anti-mouse IgG (H+L) (1/1000) (Zymed), followed by visualization of the bands on X-ray film (Kodak) using chemiluminescence (Amersham Biosciences). All steps were performed in the presence of 3% normal goat serum (Zymed).

### EXAMPLE 2

### Immunization of Mice and Hamsters with TriSpike

Balb/c mice 6-8 weeks old (n=3 per group) were immunized intraperitoneally (i.p.) with 20 μg of purified TriSpike or TriSpike in 1 mg of aluminium hydroxide gel (alum) on d0, d16 and d32. Animals in the control group received PBS with 1 mg of alum on the same days. Blood samples were collected by saphenous vein bleeding at indicated time points in accordance with local guidelines and sera were prepared and heat-inactivated.

Golden Syrian hamster 6-8 weeks old (n=4 per group) were immunized subcutaneously (s.c.) with 2, 10 or 20 μg of purified TriSpike in 2 mg of alum on d0, d21 and d42. Parallel experiment was performed in USNIH with 2, 10 or 50 μg of purified TriSpike in 1 mg of alum on the same days. Blood samples were collected by saphenous vein bleeding at indicated time points in accordance with local guidelines and sera were prepared and heat-inactivated.

### EXAMPLE 3

### Serum-Neutralization Assay

100 TCID₅₀ of SARS-CoV (strain HKU-39849) were incubated for 2 hours at 37° C. with serial 2-fold dilutions of mouse sera in quadruplicate. Virus antibody mix was then added to FRhk-4 cells in 96-well plates, and plates were incubated at 37° C. with microscopic examination for cytopathic effect (cpe) after a 4-day incubation. Neutralization titers were calculated by the Reed & Muench formula and are expressed as the reciprocal of the serum dilution which neutralized cpe in 50% of the wells (Reed & Muench, 1938).

Parallel experiment was performed in NIH NIAID. 100 TCID₅₀ of SARS-CoV (strain Urbani) were incubated for 1 hour at room temperature with serial 2-fold dilutions of hamster sera in quadruplicate. Virus antibody mix was then added to Vero cell monolayers in 96-well plates and plates were incubated at 37° C. with microscopic examination for cytopathic effect (cpe) after a 4-day incubation. The dilution of serum that completely blocked the cytopathic effect in 50% of the wells was calculated by the Reed-Muench formula (Reed & Muench, 1938).

### EXAMPLE 4

### Production of Pseudotyped Lentivirus

Recombinant retroviruses expressing a luciferase reporter gene were produced as described previously (Lozach, JBC, 2004). Briefly, 2.5×10⁶ 293T were transfected overnight using calcium phosphate method with the following plasmids: 10 μg of plasmid encoding Env-defective, luciferase-expressing, HIV-1 genome (pNL4.3.Luc R⁻E⁻pro⁻) and 10 μg of plasmid encoding codon-optimized SARS-CoV S protein. Supernatants containing SARS pseudovirus were harvested 48 hour after transfection, filtered through 0.45-μm syringe filters, aliquotted and frozen at −80° C.

### EXAMPLE 5

### ADE Assays

Twenty-five microliters of 1:250 dilutions of heat-inactivated murine or hamster sera were incubated for 1 hour at 37° C. with 25 μl of pseudovirus (50 ng p24). Fifty microliters of Raji or Daudi cells in serum-free RPMI at 2×10⁶ cell/ml were added to the Ab/pseudovirus mixture in a 96-well plate. After adsorption for 1 hour at 37° C., 100 μl of RPMI 1640 containing 5% FCS were added. The culture was refed with fresh medium 24 hour later and incubated for an additional 48 hour. The cells were washed with PBS and lysed using 20 μl of lysis reagent included in a luciferase kit (Promega). Lysates were tested for luciferase activity by the addition of 100 μl of luciferase substrate (Promega) and measured for 10 sec in a MicroBeta Jet Counter (Perkin Elmer).

### EXAMPLE 6

### SARS-CoV Challenge and Viral Load Titration

On day 56, 48 anesthetized hamsters received 100 μl (10³TCID₅₀) of SARS-CoV (Urbani strain) intranasally. Hamsters were euthanatized by lethal intraperitoneal injection with sodium pentobarbital (200 μl/hamster) on designed days. Sixteen of 48 SARS-CoV inoculated hamsters were sacrificed on days 58. Lungs were harvested and processed for viral titration. Another sixteen of 48 SARS-CoV inoculated hamsters were sacrificed on days 61. Lungs and livers were harvested and processed for pathology studies. The remaining sixteen SARS-CoV inoculated hamsters were processed for daily weighing and behavioral observation until days 77. Lungs and livers were harvested and processed for pathology studies.

### EXAMPLE 7

### Determination of Viral Titers

Tissue samples were homogenized to a final 10% (wt/vol) suspension in L15 medium with piperacillin (Sigma Aldrich Co. St louis, Mo.), gentamicin (Invitrogen, Grand Island, N.Y.), and amphotericin (Quality Biological, Gaithersburg, Md.), which were added to the tissue culture medium at final concentration of 0.4, 0.1, and 5 mg/liter, respectively. Tissue homogenates were clarified by low-speed centrifugation, and virus titers were determined in Vero cell monolayers in 24- and 96-well plates as described previously. Virus titers are expressed as TCID₅₀ per gram of tissue, with a lower limit of detection of 10^{1.5} TCID₅₀/g.

### EXAMPLE 8

### Blood Chemistry

Hamster serum samples were analyzed for the levels of AST, ALT, ALP, GGT, BUN, and total bilirubin using blood chemistry analyzer (Analytics, MedTec Lab).

### EXAMPLE 9

### Production of Immunopurified Trimeric S-Protein with Native Antigenicity for ADE and Hamster Challenge Studies

The defective Semliki Forest Virus vector coding for a full-length, codon-optimized SARS-CoV S-protein fused to a C-terminal FLAG peptide was used. Trimeric S-protein (TriSpike) was purified by immunoaffinity from transfected or infected hamster cells (BHK-21). Analysis of the apparent molecular weight of the protein by SDS-PAGE and Western Blot under non-reducing conditions revealed the predominant trimeric nature of the antigen. Higher molecular weight aggregates were occasionally observed when the protein was not heat denatured prior to SDS-PAGE.

Trimers dissociate partly into monomers when the protein is heat-denatured or DDT treated in the presence of SDS. As expected, trimers dissociate completely into monomers when heat-denatured in SDS and DTT. The trimeric and monomeric S-protein frequently migrate as doublets, which represent high-mannose glycoforms from proteins that reside in the ER at the time of lysis and glycoforms from proteins that have acquired complex N-glycans in the median-Golgi (JGV, 2005, 86, 1423-1434). Purified trimeric S-protein, have purity over 90% throughout the immunopurification procedure.

TriSpike has native antigenicity shown by reactivity with sera from 5 convalescent SARS patients by Western Blot and 11 sera tested by FACS. The native fold was further underscored by the specific binding of the TriSpike protein with soluble ACE2 receptor. Altogether these results strongly argue that purified TriSpike molecules mimick the native trimeric S-protein on the virion surface.

### EXAMPLE 10

### Antibody Dependent Enhancement from Immunized Mouse and Hamster Serum

TriSpike immunization in mice and hamster induces in vitro facilitating antibodies with some FcR expressing cell lines. The entry of SARS-CoV is mediated by the S glycoprotein, which uses the human aminopeptidase ACE2 as a functional receptor. S-mediated viral entry occurs in a pH-dependent manner and can be inhibited by S-specific sera. Pseudotyping with retroviral and lentiviral vectors has been extensively used to faithfully mimic and analyze the mechanism and the specificity of viral entry.

Previous studies demonstrated that nAbs induced by the S protein of FIPV lead to accelerated disease in vivo by the mechanism of ADE of macrophages FcR-mediated infection. This invention sought to investigate in vitro ADE of SARS-CoV entry into several FcR expressing cell lines using SARS-CoV pseudotypes. Cells were infected with pseudoviruses in the presence or absence of heat-inactivated serum from Trispike immunized mice and hamster or serum from preimmune mice and hamsters as a control. As shown in FIG. 1A, high level of transduction was detected in VeroE6 cells and pseudovirus infectivity was reduced in a dose dependent manner when preincubated with S-specific antisera. In contrast, low level of transduction was observed with J774.A1 murine macrophagic cell line cells and primary human monocyte-derived macrophages (MDM) for each experimental condition. Raji B and Daudi (not shown) EBV-transformed human B cell lines were also refractory to transduction with pseudovirus.

Unexpectedly, high level of transduction was detected when pseudoviruses were preincubated with S-specific mice and hamsters sera in a dose dependent manner (FIG. 1B & 1C). Thus, an antibody-induced shift of SARS tropism was observed, since antibodies specific to SARS Spike promotes viral entry in the otherwise refractory human Raji B cell line.

### EXAMPLE 11

### Protection of Immunized Hamsters from SARS-CoV Challenge

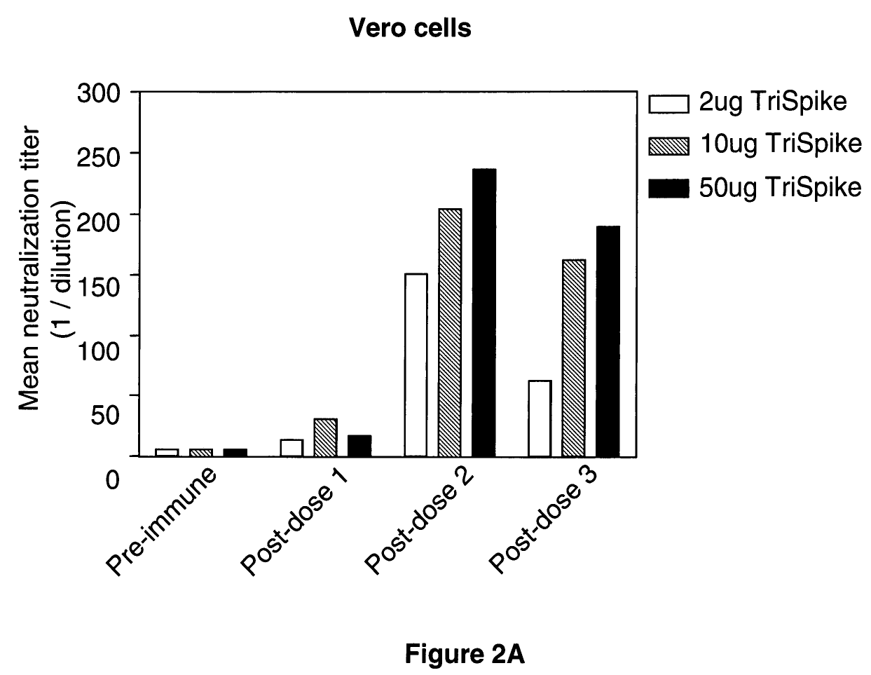

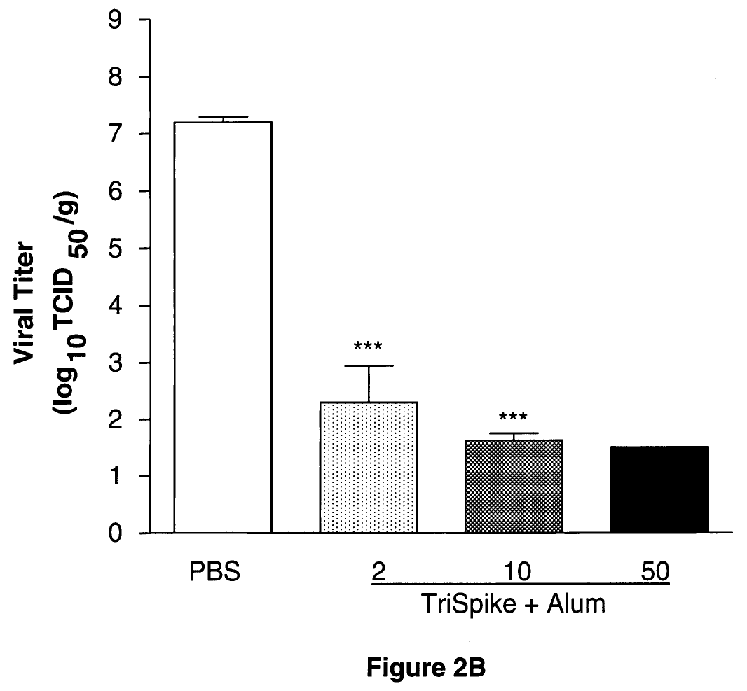

Even the neutralizing titer has at least 3 fold different; the experiment still keeps on using immunized hamster to be infected by SARS-CoV (Urbani strain). Viral replication from hamster lung tissue indicated the in vivo protective response against SARS-CoV challenge after TriSpike immunization. Results suggested the protective role of spike protein immunization from SARS-CoV challenge. 2 μg of spike protein immunization could reduce the viral load (˜100000 fold) as compared to naive hamster (FIG. 2). With higher dosage of protein immunization (10 or 50 μg), no viral load could be detected after SARS-CoV challenge (FIG. 2B). Altogether, these results suggested the ability of the antigen (recombinant trimeric spike protein) in inducing neutralizing antibody (FIG. 2A), which works against SARS-CoV both in vitro and in vivo.

### EXAMPLE 12

### Serum Markers for Hepatotoxicity

Previous study using modified vaccinia virus Ankara vector expressing SARS CoV Spike protein caused liver injury indicated the possibility in inducing organ damage. Hamster serum samples were analyzed for the levels of AST, ALT, ALP, GGT, BUN and total bilirubin. No difference was seen in TriSpike vaccinated and SARS CoV challenged hamsters compared to naive hamsters. This indicates that TriSpike vaccination did not induce hepatotoxicity in the hamster model.

10% LUNGS - Hamsters
Finalized 9 April 2005

| | | Neut titer - | Mean | 2 days p.c. | Mean | | | | | | | |
|---|---|---|---|---|---|---|---|---|---|---|---|---|
| Group | Hamster ID | pre-challenge | Neut ± SE | Viral titer | VT ± SE | 24 well | 0 | 1 | 2 | 3 | 4 | 5 |
| PBS | 2367 | <1:8 | <1:8 ± 0.0 | 7.20 | 7.2 ± 1 | ++ | cccc | ++++ | ++++ | ++++ | −+++ | −+−− |
| PBS | 2368 | <1:8 | | 7.45 | | ++ | cccc | ++++ | ++++ | ++++ | ++++ | −−+− |
| PBS | 2369 | <1:8 | | 6.95 | | ++ | cccc | ++++ | ++++ | ++++ | +++− | −−−− |
| PBS | 2370 | <1:8 | | 7.20 | | ++ | cccc | ++++ | ++++ | ++++ | ++++ | −−−− |
| 2 μg | 2371 | 64 | 63 ± 10 | 4.20 | 2.3 ± .6 | ++ | cccc | ++++ | −−−− | −−−− | −−−− | −−−− |
| 2 μg | 2372 | 51 | | 2.00 | | +− | −−−− | −−−− | −−−− | −−−− | −−−− | −−−− |
| 2 μg | 2373 | 45 | | 1.50 | | −− | −−−− | −−−− | −−−− | −−−− | −−−− | −−−− |
| 2 μg | 2374 | 91 | | 1.50 | | −− | −−−− | −−−− | −−−− | −−−− | −−−− | −−−− |
| 10 μg | 2375 | 51 | 162 ± 82 | 1.50 | 1.6 ± .1 | −− | −−−− | −−−− | −−−− | −−−− | −−−− | −−−− |
| 10 μg | 2376 | 406 | | 1.50 | | −− | −−−− | −−−− | −−−− | −−−− | −−−− | −−−− |
| 10 μg | 2377 | 102 | | 1.50 | | −− | −−−− | −−−− | −−−− | −−−− | −−−− | −−−− |
| 10 μg | 2378 | 91 | | 2.00 | | +− | −−−− | −−−− | −−−− | −−−− | −−−− | −−−− |
| 50 μg | 2379 | 256 | 190 ± 59 | 1.50 | <1.5 ± 0.0 | −− | −−−− | −−−− | −−−− | −−−− | −−−− | −−−− |
| 50 μg | 2380 | 323 | | 1.50 | | −− | −−−− | −−−− | −−−− | −−−− | −−−− | −−−− |
| 50 μg | 2381 | 91 | | 1.50 | | −− | −−−− | −−−− | −−−− | −−−− | −−−− | −−−− |
| 50 μg | 2382 | 91 | | 1.50 | | −− | −−−− | −−−− | −−−− | −−−− | −−−− | −−−− |
| | beck titer | | | | | | ++++ | ++++ | −+−− | +−−− | −−−− | −−−− |

| | | | | | | | | | Mann-Whitney | t-test | |
|---|---|---|---|---|---|---|---|---|---|---|---|
| 6 | 7 | 8 | 9 | TCID50 | TCID50/g | assigning 1.5 | mean | se | p value | p value | |
| −−−− | −−−− | −−−− | −−−− | 4.50 | 7.20 | 7.20 | 7.20 | 0.10 | | | PBS |
| −−−− | −−−− | −−−− | −−−− | 4.75 | 7.45 | 7.45 | | | | | PBS |
| −−−− | −−−− | −−−− | −−−− | 4.25 | 6.95 | 6.95 | | | | | PBS |
| −−−− | −−−− | −−−− | −−−− | 4.50 | 7.20 | 7.20 | | | | | PBS |
| −−−− | −−−− | −−−− | −−−− | 1.50 | 4.20 | 4.20 | 2.30 | 0.64 | | | 2 μg |
| −−−− | −−−− | −−−− | −−−− | | 2.00 | 2.00 | | | | | 2 μg |
| −−−− | −−−− | −−−− | −−−− | | <1.5 | 1.50 | | | | | 2 μg |
| −−−− | −−−− | −−−− | −−−− | | <1.5 | 1.50 | | | | | 2 μg |
| −−−− | −−−− | −−−− | −−−− | | <1.5 | 1.50 | 1.63 | 0.13 | | | 10 μg |
| −−−− | −−−− | −−−− | −−−− | | <1.5 | 1.50 | | | | | 10 μg |
| −−−− | −−−− | −−−− | −−−− | | <1.5 | 1.50 | | | | | 10 μg |
| −−−− | −−−− | −−−− | −−−− | | 2.00 | 2.00 | | | | | 10 μg |
| −−−− | −−−− | −−−− | −−−− | | <1.5 | 1.50 | 1.50 | 0.00 | | | 50 μg |
| −−−− | −−−− | −−−− | −−−− | | <1.5 | 1.50 | | | | | 50 μg |
| −−−− | −−−− | −−−− | −−−− | | <1.5 | 1.50 | | | | | 50 μg |
| −−−− | −−−− | −−−− | −−−− | | <1.5 | 1.50 | | | | | 50 μg |

### REFERENCES

The following publications are cited herein. The entire disclosure of each publication is relied upon and the entire disclosure of each publication is incorporated by reference herein:
1. Chen, Z., L. Zhang, C. Qin, L. Ba, C. E. Yi, F. Zhang, Q. Wei, T. He, W. Yu, J. Yu, H. Gao, X. Tu, A. Gettie, M. Farzan, K. Y. Yuen, and D. D. Ho. 2005. Recombinant modified vaccinia virus Ankara expressing the spike glycoprotein of severe acute respiratory syndrome coronavirus induces protective neutralizing antibodies primarily targeting the receptor binding region. J Virol 79:2678-88.
2. Czub, M., H. Weingarti, S. Czub, R. He, and J. Cao. 2005. Evaluation of modified vaccinia virus Ankara based recombinant SARS vaccine in ferrets. Vaccine 23:2273-9.
3. He, Y., Q. Zhu, S. Liu, Y. Zhou, B. Yang, J. Li, and S. Jiang. 2005. Identification of a critical neutralization determinant of severe acute respiratory syndrome (SARS)-associated coronavirus: importance for designing SARS vaccines. Virology 334:74-82.
4. Johnston, R. E. 2004. A candidate vaccine for severe acute respiratory syndrome. N Engl J Med 351:827-8.
5. Nal, B., C. Chan, F. Kien, L. Siu, J. Tse, K. Chu, J. Kam, I. Staropoli, B. Crescenzo-Chaigne, N. Escriou, S. van der Werf, K. Y. Yuen, and R. Altmeyer. 2005. Differential maturation and subcellular localization of severe acute respiratory syndrome coronavirus surface proteins S, M and E. J Gen Virol 86:1423-34.
6. Roberts, A., L. Vogel, J. Guarner, N. Hayes, B. Murphy, S. Zaki, and K. Subbarao. 2005. Severe acute respiratory syndrome coronavirus infection of golden Syrian hamsters. J Virol 79:503-11.
7. Weingartl, H., M. Czub, S. Czub, J. Neufeld, P. Marszal, J. Gren, G. Smith, S. Jones, R. Prouix, Y. Deschambault, E. Grudeski, A. Andonov, R. He, Y. Li, J. Copps, A. Grolla, D. Dick, J. Berry, S. Ganske, L. Manning, and J. Cao. 2004. Immunization with modified vaccinia virus Ankara-based recombinant vaccine against severe acute respiratory syndrome is associated with enhanced hepatitis in ferrets. J Virol 78:12672-6.
8. Yang, Z. Y., W. P. Kong, Y. Huang, A. Roberts, B. R. Murphy, K. Subbarao, and G. J. Nabel. 2004. A DNA vaccine induces SARS coronavirus neutralization and protective immunity in mice. Nature 428:561-4.
9. Zhou, J., W. Wang, Q. Zhong, W. Hou, Z. Yang, S. Y. Xiao, R. Zhu, Z. Tang, Y. Wang, Q. Xian, H. Tang, and L. Wen. 2005. Immunogenicity, safety, and protective efficacy of an inactivated SARS-associated coronavirus vaccine in rhesus monkeys. Vaccine 23:3202-9.
10. Zhou, T., H. Wang, D. Luo, T. Rowe, Z. Wang, R. J. Hogan, S. Qiu, R. J. Bunzel, G. Huang, V. Mishra, T. G. Voss, R. Kimberly, and M. Luo. 2004. An exposed domain in the severe acute respiratory syndrome coronavirus spike protein induces neutralizing antibodies. J Virol 78:7217-26.

What is claimed is:

1. An immunogenic composition comprising at least one recombinant SARS CoV Spike protein having all of the epitopes of native Spike protein and free of other native SARS CoV components in an amount sufficient to induce an immunogenic or protecting response in vivo, and a pharmaceutically acceptable carrier therefor.

2. The immunogenic composition of claim 1, wherein said composition comprises a neutralizing amount of at least one SARS CoV Spike protein.

3. The immunogenic composition according to claims 1 and 2, further comprising an alum adjuvant.

4. A method of using the immunogenic composition of claim 1, comprising administering said immunogenic composition to a host in an amount sufficient to induce an immunogenic or protecting response in vivo.

5. The method according to claim 4, wherein the immunogenic composition is administered by oral, respiratory, or parenteral routes.

6. The method according to claim 4, wherein the immunogenic composition comprises an alum adjuvant.

7. A method of vaccinating against SARS CoV comprising administering any combination of the nucleic acid encoding Spike polypeptides, the proteins and polypeptides to an individual.

8. The method of vaccinating according to claim 7, wherein the individual is a mammal.
